(19) United States
(12) Patent Application Publication
(10) Pub. No.: US 2018/0271710 A1
(43) Pub. Date: Sep. 27, 2018

(54) Wireless earpiece for tinnitus therapy

(71) Applicant: BRAGI GmbH, Munchen (DE)

(72) Inventors: Peter Vincent Boesen, Munchen (DE); Darko Dragicevic, Munchen (DE); Felix Hägele, Munchen (DE)

(73) Assignee: BRAGI GmbH, Munchen (DE)

(21) Appl. No.: 15/906,867

(22) Filed: Feb. 27, 2018

Related U.S. Application Data
(60) Provisional application No. 62/474,993, filed on Mar. 22, 2017.

(51) Int. Cl. A61F 11/00; H04R 1/22; A61B 5/12; A61B 5/00

(52) U.S. Cl. CPC A61F 11/00; H04R 1/22; A61B 5/128; A61B 5/0004; H04R 25/75; A61B 5/7203; H04R 2420/07; A61F 2250/0002; A61B 5/6817

(57) ABSTRACT

A wireless earpiece in embodiments of the present invention may have one or more of the following features: (a) a housing for fitting in an ear of a user, (b) a logic engine controlling functionality of the wireless earpiece, (c) a plurality of sensors reading user input from the user, and (d) a transceiver communicating with at least a wireless device, wherein the logic engine receives a selection of audio through the plurality of sensors, determines whether a tinnitus frequency associated with a user wearing the wireless earpiece is present in the audio, filters the tinnitus frequency from the audio to generate filtered audio, and plays the filtered audio to the user utilizing one or more speakers.

### PRIORITY STEPMENT

This application claims priority to U.S. Provisional Patent Application No. 62/474,993 filed on Mar. 22, 2017 titled Wireless Earpiece for Tinnitus Therapy all of which is hereby incorporated by reference in its entirety.

### FIELD OF THE INVENTION

The illustrative embodiments relate to wearable devices. Particularly, illustrative embodiments of the present invention relate to wireless earpieces. More particularly, but not exclusively, the illustrative embodiments relate to wireless earpieces for tinnitus therapy.

### BACKGROUND

Tinnitus is the sensation of noise caused by a bodily condition, such as disturbance of the auditory nerve or other neurological pathology. Absent of external auditory stimulus, tinnitus patients often hear one or more tones. The causes of tinnitus are believed to be numerous and still not quite fully understood. Typical tinnitus therapy includes drug therapy and/or sound or masking therapy, such as residual inhibition therapy.

Physicians use various testing procedures to determine the parameters of the tinnitus and the tones to be applied to the patient for specific types of therapy. A physician can perform an audible diagnostic test with qualitative patient feedback. Other testing procedures also include the use of expensive functional magnetic resonance imaging (FMRI), positron emission tomography (PET), electro-encephalograms (EEGs), Auditory event-related potential (ERP) and magnetic stimulation.

During sound therapy the patient is exposed to specific tones determined as a function of the tinnitus tones heard by the patient and any tones of which the patient has partial or total hearing loss. The tones used during sound therapy are intended to reduce the tinnitus symptoms. However, most effects from a single session of sound therapy are short lived, requiring patients to undergo repeated therapy sessions. In a few cases, fifteen minutes of residual inhibition therapy relieved tinnitus symptoms for a single day. Tinnitus symptoms vary significantly, not just from patient to patient, but also over time, with or without therapy, and over the course of therapy. Similarly, the therapeutic sounds (tones and volumes) producing the best results vary from patient to patient, and the most effective therapeutic sounds often vary over the course of extended treatment.

The lack of effective sound therapies and flexible audiological platforms inhibits physicians from prescribing the most effective treatments for tinnitus. Furthermore, physicians must regularly test patients' progress to determine how to adjust the sound therapy. A large quantity of the physician's and patient's time and resources are used amending the prescribed therapies. This need for re-diagnosis also often leads to outdated, and thus imprecise, prescribed therapies, and results in increased patient absenteeism for return visits to be re-evaluated and have sound therapies updated.

The ability to track patient treatment for sound therapy can be difficult, especially when the sound therapy occurs outside of a physician's office. The patient can be non-compliant by not following the sound treatment protocols. This lack of accurate tracking of the applied therapy impairs physicians' abilities to treat patients properly. The lack of accurate tracking also impedes health professionals attempting to create an accurate tinnitus model to use for predicting the most effective therapies and/or custom modification of an individual's therapy.

Therefore, there exists a need for a combined system to provide tinnitus therapy with frequent re-evaluation of the tinnitus profile. There also exists a need for an evaluation system reducing or obviating physician visits. There is also a need for an easy to transport therapeutic and evaluation device for use by the patient. Furthermore, there is a need for an accurate empirical model of tinnitus, such as a model accurately predicting an effective sound therapy based on diagnostic inputs.

Current tinnitus therapies provide patients who already have a tinnitus diagnosis with therapeutic white noise and other forms of audio. However, patients often must pay high fees, visit specialists, and use expensive and complex equipment to get a diagnosis. After obtaining a tinnitus diagnosis specifying the audio frequency at which the tinnitus occurs, patients are limited in their therapeutic audio options. Therefore, what is needed is a convenient, individualized tool for diagnosing, administering, and tracking tinnitus therapy.

### SUMMARY

Therefore, it is a primary object, feature, or advantage of the illustrative embodiments to improve over the step of the art.

A method for treating tinnitus utilizing wireless earpieces in embodiments of the present invention may have one or more of the following steps: (a) receiving a selection of audio through the wireless earpieces, (b) determining whether a tinnitus frequency associated with a user wearing the wireless earpieces is present in the audio, (c) filtering the tinnitus frequency from the audio to generate filtered audio, (d) playing the filtered audio to the user utilizing the wireless earpieces, (e) initiating a tinnitus test for the user, (f) playing tones associated with the tinnitus test to the user, (g) determining whether the tinnitus frequency is played to the user, (h) associating the tinnitus frequency with the user in response to determining the tinnitus frequency was played to the user, (i) distributing the tinnitus frequency to one or more devices in communication with the wireless earpieces, (j) tracking filtering of the tinnitus frequency performed by the wireless earpieces, (k) receiving user input confirming the tinnitus frequency, and (l) identifying the user utilizing the wireless earpieces.

A wireless earpiece in embodiments of the present invention may have one or more of the following features: (a) a housing for fitting in an ear of a user, (b) a logic engine controlling functionality of the wireless earpiece, (c) a plurality of sensors reading user input from the user, and (d) a transceiver communicating with at least a wireless device, wherein the logic engine receives a selection of audio through the plurality of sensors, determines whether a tinnitus frequency associated with a user wearing the wireless earpiece is present in the audio, filters the tinnitus frequency from the audio to generate filtered audio, and plays the filtered audio to the user utilizing one or more speakers.

A method for treating tinnitus utilizing wireless earpieces in embodiments of the present invention may have one or more of the following steps: (a) receiving ambient sound through the wireless earpieces, (b) determining whether a tinnitus frequency has been associated with a user, (c) initiating a tinnitus test for the user if no tinnitus frequency has been associated with the user, (d) determining whether the tinnitus frequency associated with a user wearing the wireless earpieces is present in the ambient sound if the tinnitus frequency has been associated with the user, (e) filtering the tinnitus frequency from the ambient sound to generate filtered ambient sound, (f) playing the filtered ambient sound to the user utilizing the wireless earpieces, (g) playing tones associated with the tinnitus test to the user, (h) determining whether the tinnitus frequency is played to the user, and (i) associating the tinnitus frequency with the user in response to determining the tinnitus frequency was played to the user.

One or more of these and/or other objects, features, or advantages of the illustrative embodiments will become apparent from the specification and claims follow. No single embodiment need provide every object, feature, or advantage. Different embodiments may have different objects, features, or advantages. Therefore, the illustrative embodiments are not to be limited to or by any objects, features, or advantages stated herein.

### BRIEF DESCRIPTION OF THE DRAWINGS

FIG. 1 illustrates tinnitus therapy system with wireless earpieces configured to determine a user's tinnitus frequency in accordance with an illustrative embodiment;

FIG. 2 is a block diagram of wireless earpiece configured to perform tinnitus therapy in accordance with an illustrative embodiment;

FIG. 3 is a flowchart of a process for associating a tinnitus frequency with a user in accordance with an illustrative embodiment;

FIG. 4 is a flowchart of a process for treating tinnitus in accordance with an illustrative embodiment; and

FIG. 5 includes a flowchart of one implementation of the method of adding an ambient environment sound and treating tinnitus in an earpiece in accordance with an illustrative embodiment.

### DETAILED DESCRIPTION

The following discussion is presented to enable a person skilled in the art to make and use the present teachings. Various modifications to the illustrated embodiments will be clear to those skilled in the art, and the generic principles herein may be applied to other embodiments and applications without departing from the present teachings. Thus, the present teachings are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings. While embodiments of the present invention are discussed in terms of wearable electronic devices, it is fully contemplated embodiments of the present invention could be used in most any wireless earpieces without departing from the spirit of the invention.

It is an object, feature, or advantage of the illustrative embodiments to provide a wireless earpiece which is configured for determining the audio frequency causing the user discomfort.

It is a still further object, feature, or advantage of the illustrative embodiments to diagnose the audio frequency as a tinnitus frequency.

Another object, feature, or advantage is to block or attenuate the tinnitus frequency from a first audio source.

A further object, feature, or advantage is to allow the user to store and replay tinnitus filtered audio from the wireless earpiece or other electronic devices.

Yet another object, feature, or advantage is to create a tinnitus therapy by blocking tinnitus frequencies from any audio sources selected by the user and providing the user with wireless earpieces for playing the filtered audio.

Still another object, feature, or advantage is to track the user or patient's use of the tinnitus therapy using an application.

A system, method, and wireless earpieces for treating tinnitus is disclosed in detail below. A selection of audio is received through the wireless earpieces. A determination is made whether a tinnitus frequency is associated with a user wearing the wireless earpieces. The tinnitus frequency is filtered from the audio to generate filtered audio. The filtered audio is played to the user utilizing the wireless earpieces. Another embodiment provides a processor for executing a set of instructions. The set of instructions may be stored in a memory and executed to perform the method herein described.

In an alternative embodiment, the wireless earpieces initiate a tinnitus test for the user, play tones associated with the tinnitus test to the user, determine whether the tinnitus frequency is played to the user, and associate the tinnitus frequency with the user in response to determining the tinnitus frequency was played to the user.

According to one aspect, a wireless earpiece(s) is configured to determine the frequency at which a user hears a sound without an identifiable source (the tinnitus frequency), and to filter this frequency out of the user's selected audio sources. The wireless earpiece or set of earpieces includes a wireless earpiece housing, a tinnitus diagnosis system, and a therapeutic filtering system. The audio source may include different tones. When the user hears a tone matching his or her perceived tinnitus tone, the user can confirm the tone. After the user confirms the tone, the frequency of tone is transmitted to the therapeutic filtering system. The tone may be transmitted using a linear continuous-time filter, or another type of filter. The filter may be integrated with logic of the wireless earpieces to limit the bandwidth of the output signal to the band allocated for transmission. The filter may also work with the logic or a transceiver to allow signals within a select range of frequencies to be heard, thereby filtering out the tinnitus frequency. After the filter has removed the tinnitus tone from the user's desired audio source, the audio source may be stored on the earpiece or in another location, so the user can replay it. The filtered audio source may need to be amplified and then transformed into a digitally encoded signal to be stored in memory. The user may also filter new streams of audio without storing those first audio sources. The user can track how often he or she is practicing the tinnitus therapy by downloading software to the earpiece which communicates with an application. According to user presets, each time the user listens to an audio source, the duration of time, type of audio, volume, etc., may be sent to the application or tracked by the user via the application. The application may be located on a different device.

When a person hears a sound when no sounds are present, he or she may suffer from and be diagnosed with tinnitus. The tinnitus may be objective or subjective. Objective tinnitus is actual noise generated by a physiological phenomenon occurring near the middle ear. Sometimes a health care provider treating the patient with tinnitus can hear the noise created by objective tinnitus. Subjective tinnitus involves noise only the patient can hear. Patients with tinnitus often characterize the noise as a ringing sound.

Tinnitus may also be muscular, created by myoclonus (involuntary spasms caused by muscular contractions) or pulsatile. Pulsatile tinnitus synchronizes the ringing sound with the patient's heartbeat. The illustrative embodiments may help individuals suffering from subjective tinnitus. Several factors may lead to subjective tinnitus including: aging, hearing loss, medications, head or neck trauma, exposure to loud noise, smoking, or cardiovascular problems. One form of treatment for tinnitus uses lateral inhibition. Lateral inhibition is the capacity of an excited neuron to reduce the activity of its neighbors. The goal of tinnitus treatments using lateral inhibition is to block out an audio frequency at which the tinnitus is occurring. By exposing the patient (or user) to audio blocking or filtering out the tinnitus ringing noise, a “blocking frequency,” the neighboring neurons of tinnitus-accustomed neurons are stimulated. Over time, as the user listens to the filtered audio, the neighboring neurons reduce the activity of the tinnitus-accustomed neurons. When the tinnitus-accustomed neurons are no longer stimulated, the user no longer perceives a sound without an identifiable source or hears a ringing sound when there is none. Diligent, repeated use of lateral inhibition therapy may help to decrease tinnitus symptoms more quickly.

Lateral inhibition may be performed by the wireless earpieces. For example, the processor or other logic of the wireless earpieces may implement processes with the speakers to treat tinnitus. The patient also has the option of downloading software to the wireless earpiece working with an application on a remote device and an audio intelligence center to track the patient's use of tinnitus therapy. As the patient continues to use the tinnitus therapy, their experience of tinnitus lessens. Depending on the individual patient and their use of the therapy, the experience of tinnitus can completely dissipate.

The illustrative embodiments utilize a convenient wireless earpiece to provide patients with the tools to determine the frequency at which the tinnitus is occurring, the ability to filter their desired audio to generate a therapeutic frequency for the patient, and the capacity to track their therapy use on an app. All these tools are conveniently located within a wireless earpiece. The patient may use another electronic device, including but not limited to, a smartphone, computer, tablet, or other device to set the user settings for the earpiece and to chart out and view the patient's progress in therapy. In one embodiment of the tinnitus therapy utilizing wireless earpieces, a user listens to different tones (first audio sources) and confirms the similarity of these tones to the tinnitus noise she has been hearing to produce a tinnitus frequency. When the user's tinnitus frequency is determined, the determined frequency may then be filtered out of the user's first audio sources.

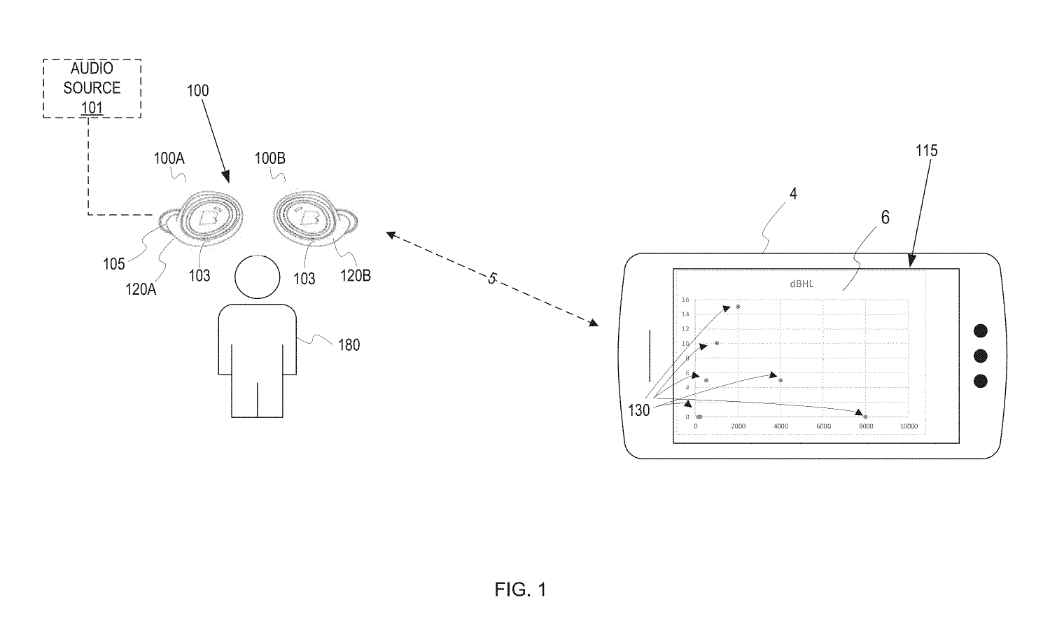

FIG. 1 displays a user 180 wearing a left wireless earpiece 100A and a right wireless earpiece 100B in accordance with an illustrative embodiment. The left wireless earpiece 100A and the right wireless earpiece 100B are individually or jointly the wireless earpieces 100 or wireless earpiece 100. Each wireless earpiece 100A, 100B also has a left earpiece housing 120A and a right earpiece housing 120B.

In one embodiment, the wireless earpieces 100 includes a housing 103 shaped to fit substantially within the ears of a user 180. The housing 103 is a support structure at least partially enclosing and housing the electronic components of the wireless earpieces 100. The housing 103 may be composed of a single structure or multiple intercoupled structures. An exterior portion of the wireless earpieces 100 may include any number of sensors, such as infrared sensors. The infrared sensors may include emitter and receivers detecting and measuring infrared light radiating from objects in their field of view. The infrared sensors may detect gestures, touches, or other user input against an exterior portion of the wireless earpieces 100 visible when worn by the user 180. The infrared sensors may also detect infrared light or motion. The infrared sensors may be utilized to determine whether the wireless earpieces 100 are being worn, moved, approached by a user, set aside, stored in a smart case, placed in a dark environment, or so forth. The sensors may also be integrated in the housing 103 or any other portion of the wireless earpieces 100.

The housing 103 defines an extension 105 configured to fit substantially within the ear of the user 180. The extension 105 may include one or more speakers or vibration components for interacting with the user 180. The extension 105 may be removably covered by one or more sleeves. The sleeves may be changed to fit the size and shape of the user's ears. The sleeves may come in various sizes and have extremely tight tolerances to fit the user 180 and one or more other users may utilize the wireless earpieces 100 during their expected lifecycle. In another embodiment, the sleeves may be custom built to support the interference fit utilized by the wireless earpieces 100 while also being comfortable when worn. The sleeves are shaped and configured to not cover various sensor devices of the wireless earpieces 100. Separate sleeves may be utilized if different users are wearing the wireless earpieces 100. The sleeves may also represent foam plugs.

Each of the wireless earpieces 100 may be utilized to play music or audio, track user biometrics, perform communications (e.g., two-way, alerts, etc.), provide feedback/input, or any number of other tasks. The wireless earpieces 100 may manage execution of software or sets of instructions stored in an on-board memory of the wireless earpieces 100 to accomplish numerous tasks. The wireless earpieces 100 may also be utilized to control, communicate, manage, or interact with several other computing, communications, or wearable devices, such as smart phones, laptops, personal computers, tablets, holographic displays, virtual reality systems, gaming devices, projection systems, vehicles, smart glasses, helmets, smart glass, watches or wrist bands, chest straps, implants, displays, clothing, or so forth.

In one embodiment, the wireless earpieces 100 may be integrated with, control, or otherwise communicate with a personal area network. A personal area network is a network for data transmissions among devices, such as personal computing, communications, camera, vehicles, entertainment, and medical devices. The personal area network may utilize any number of wired, wireless, or hybrid configurations and may be stationary or dynamic. For example, the personal area network may utilize wireless network protocols or standards, such as INSTEON, IrDA, Wireless USB, near field magnetic induction (NFMI), Bluetooth, Z-Wave, ZigBee, Wi-Fi, ANT+ or other applicable radio frequency signals. In one embodiment, the personal area network may move with the user.

As noted, the wireless earpieces 100 may include any number of sensors for reading user biometrics, such as pulse rate, blood pressure, blood oxygenation, temperature, orientation, calories expended, blood or sweat chemical content, voice and audio output, impact levels, and orientation (e.g., body, head, etc.). The sensors may also determine the user's location, position, velocity, impact levels, and so forth. The sensors may also receive user input and convert the user input into commands or selections made across the personal devices of the personal area network. For example, the user input detected by the wireless earpieces 100 may include voice commands, head motions, finger taps, finger swipes, motions or gestures, or other user inputs sensed by the wireless earpieces 100. The user input may be received, parsed, and converted into commands associated with the input utilized internally by the wireless earpieces 100. The wireless earpieces 100 may perform sensor measurements for the user to read any number of user biometrics. The user biometrics may be analyzed including measuring deviations or changes of the sensor measurements over time, identifying trends of the sensor measurements, and comparing the sensor measurements to control data for the user. The sensors may also perform ear mapping of the user to best configure the components for the tinnitus treatment process.

The wireless earpieces 100 may also measure environmental conditions, such as temperature, location, barometric pressure, humidity, radiation, wind speed, and other applicable environmental data. The wireless earpieces 100 may also communicate with external devices to receive additional sensor measurements. The wireless earpieces 100 may communicate with external devices to receive available information, which may include information received through one or more networks, such as the Internet. The detection of biometrics and environmental information may be enhanced utilizing each of the wireless earpieces 100. In addition, the separate measurements may be utilized for mapping or otherwise distinguishing applicable information. The environmental conditions and information may be projected by the user based on a user selection, automated process, user preferences, or so forth.

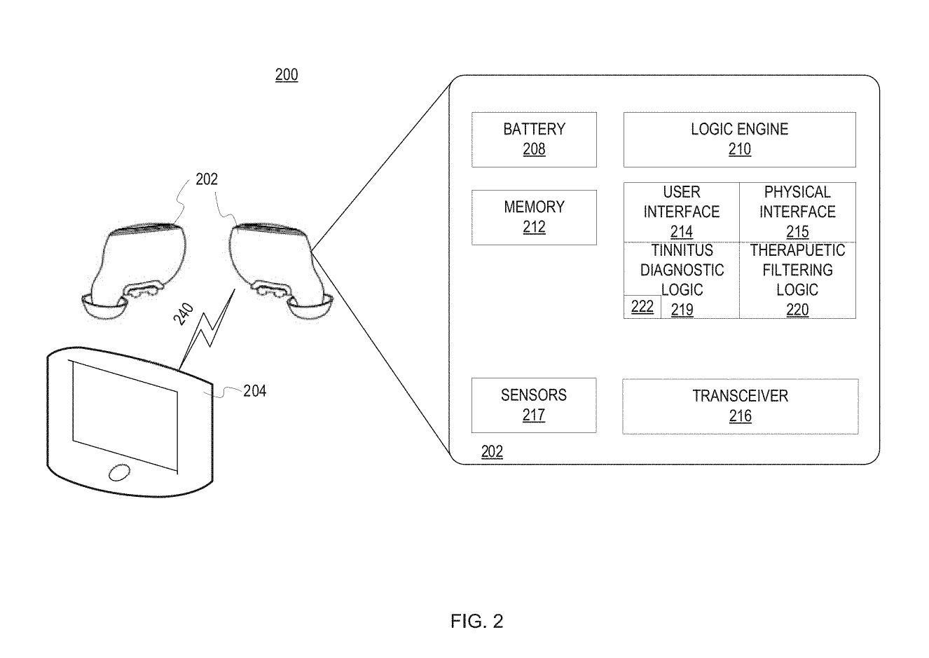

In one embodiment, the user listens to a first audio source 101. The first audio source 101 is processed via the tinnitus diagnostic system 115, described in further detail in FIG. 2. In one embodiment, the first audio source 101 may represent communications from an external wireless device 4. For example, the wireless earpieces 100 may be linked with the wireless device 4 utilizing a Bluetooth, Wi-Fi, or similar connection 5. In another embodiment, the first audio source 101 may represent a tinnitus diagnostic application executed by the wireless earpieces 100 on tones played in a pre-defined order to find tones affecting the user 180. In another embodiment, the first audio source 101 may represent ambient sound in which a tinnitus diagnostic application is executed by the wireless earpieces 100 executing an audio transparency system able to capture the world around the user.

In one embodiment, the wireless earpieces 100 broadcast the first audio source 101 including several audio tones. The user 180 listens to the first audio source 101 and confirms or rejects the first audio source 101 as matching the audio tone at which the user 180 suffers from tinnitus. The audio tone at which the individual user suffers from tinnitus is referred to herein as the tinnitus frequency 130. In one embodiment, the tone may be increased or decreased until the tinnitus frequency 130 is found. The user may acknowledge the tinnitus frequency 130 utilizing a verbal command, gesture, head nod, or so forth.

In one embodiment, one or more of the wireless earpieces 100 may be placed in a discovery mode to find the tinnitus frequency 130. The discovery mode may represent an automated process including verbal, textual, or other instructions communicated through the wireless earpieces 100 as well as the wireless device 4. In another embodiment, the discovery mode may represent a manual process where the audio source 101 varies tones based on feedback from the user 180 (e.g., “is this the frequency where you experience tinnitus if yes nod your head up and down, if no nod your head side to side”) with the user selecting when to change the tones. When the first audio source 101 matches the tinnitus frequency 130, the user 180 may proceed to save this tinnitus frequency 130 on one or more of the wireless earpieces 100 or the wireless device 4. The tinnitus frequency 130 may be associated with applicable information, such as date, time, identified user, ear or ears tested, and so forth. The tinnitus frequency 130 may also, upon confirmation by the user, be processed by the therapeutic filtering system 220 of FIG. 2 and used to filter the first audio source 101. The user 180 also has the option of viewing the results of her first audio source test on the wireless device 4. FIG. 1 displays an example of test results 6 from the first audio source on the wireless device 4. The wireless device 4 may include, but is not limited to, an electronic device, a tablet, a laptop, a gaming device, a fitness tracker, a smartphone, an audiogram, and so forth. The test results 6 may be sent to one or more devices or electronics. In one embodiment, the test results 6 are sent to a medical professional working with the user 180.

FIG. 2 displays a block diagram of a wireless earpiece 202 configured to perform tinnitus therapy in accordance with an illustrative embodiment. As previously noted, the wireless earpieces 202 may be referred to or described herein as a pair (wireless earpieces) or singularly (wireless earpiece). The description may also refer to components and functionality of each of the wireless earpieces 202 collectively or individually. In one embodiment, the wireless earpiece system 200 may enhance communications and functionality of the wireless earpieces 202. In one embodiment, the wireless earpiece system 200 or wireless earpieces 202 may communicate directly or through one or more networks 240 (e.g., Wi-Fi, mesh networks, cell networks, etc.).

A first audio source 204 enters from outside of the wireless earpieces 202. The first audio source 204 may be transmitted from a tablet, smart phone, or another electronic device to the wireless earpieces 202 through a wireless connection, link or signal. In another example, the first audio source 204 may be outside noise or a person speaking. The first audio source 204 may communicate real-time data, in-application playback, discrete messages, recorded content, environmental sounds and audio, or so forth. In one embodiment, the first audio source 204 may communicate several different tones presented to the user. The first audio source 204 may also represent a gaming device, tablet computer, vehicle system (e.g., GPS, speedometer, pedometer, entertainment system, etc.), media device, smart watch, laptop, smart glass, radio or other electronic devices or input devices available to the wireless earpieces 202. User input, commands, and communications may be received from either the wireless earpieces 202 or the first audio source 204 for implementation on either of the devices of the wireless earpiece system 200 (or other externally coupled devices).

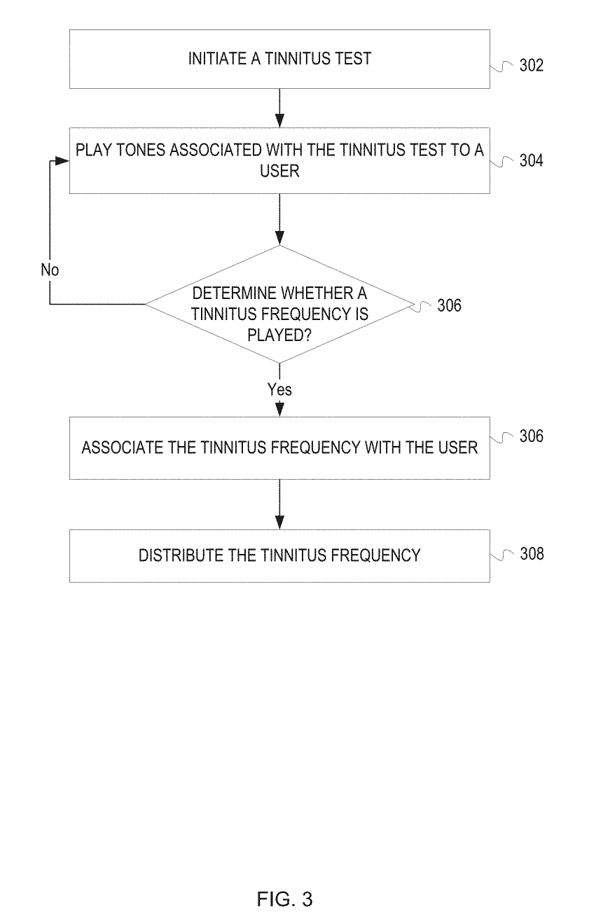

In one embodiment, a tinnitus diagnostic logic 219 may be activated to determine a tinnitus frequency 222 associated with the user. For example, the user listens to the tones to determine which tone most closely resembles the tone of her individual tinnitus frequency. The user 180 may give feedback via sensors 217. As noted, the sensors 217 may include microphones, pulse oximeters, accelerometers, thermometers, barometers, radiation detectors, gyroscopes, magnetometers, global positioning systems, beacon detectors, inertial sensors, photo detectors, miniature cameras, optical/infrared sensors, contact sensors, and other similar instruments. The sensors 217 may sense voice, gesture, touch, or other input to control, manage, or interact with the wireless earpieces. If the feedback confirms the tone matches the user's individual tinnitus frequency 222, the tinnitus frequency 222 may be sent to a memory 212 or other storage component of the wireless earpiece 100. The tinnitus frequency 130 may also be sent to the therapeutic filtering system 220 as well as the first audio source 204. Further details concerning the therapeutic filtering system 220 is given in FIG. 3.

In some embodiments, the first audio source 204 may act as a logging tool for receiving information, data, or measurements made by the wireless earpieces 202 together or separately. For example, the first audio source 204 may receive or download biometric data from the wireless earpieces 202 in real-time for users utilizing the wireless earpieces 202. For example, the tinnitus frequency 222 may be determined by the tinnitus diagnostic logic 219 and utilized by the therapeutic filtering logic 220. As a result, the first audio source 204 may be utilized to store, display, and synchronize data for the wireless earpieces 202 as well as manage communications. For example, the first audio source 204 may display pulse, proximity, location, oxygenation, distance, calories burned, and so forth as measured by the wireless earpieces 202. The first audio source 204 may be configured to receive and display an interface, selection elements, and alerts indicate conditions for diagnosing tinnitus frequencies 222 and filtering those tinnitus frequencies 222. For example, the wireless earpieces 202 may utilize factors, such as changes in motion or light, distance thresholds between the wireless earpieces 202 and/or first audio source 204, signal activity, user orientation, user speed, user location, environmental factors (e.g., temperature, humidity, noise levels, proximity to other users, etc.) or other automatically determined or user specified measurements, factors, conditions, or parameters to implement various features, functions, and commands.

The first audio source 204 may also include any number of optical sensors, touch sensors, microphones, and other measurement devices (sensors 217) providing feedback or measurements the wireless earpieces 202 may utilize to determine an appropriate mode, settings, or enabled functionality. The wireless earpieces 202 and the first audio source 204 may have any number of electrical configurations, shapes, and colors and may include various circuitry, connections, and other components.

In one embodiment, one or both wireless earpieces 202 may include a battery 208, a logic engine 210, a memory 212, a user interface 214, a physical interface 215, a transceiver 216, and sensors 217. The first audio source 204 may have any number of configurations and include components and features like the wireless earpieces 202 as are known in the art. In one embodiment, the tinnitus diagnostic logic 219 and the therapeutic filtering logic 220 may be integrated with the logic engine 210. In other embodiments, the tinnitus diagnostic logic 219 and the therapeutic filtering logic 220 represent separate logic, chips, hardware, specialized software, and so forth. The tinnitus diagnostic and treatment functions and modes may also be implemented as part of the logic engine 210, user interface, or other hardware, software, or firmware of the wireless earpieces 202 and/or first audio source 204.

The battery 208 is a power storage device configured to power the wireless earpieces 202. In other embodiments, the battery 208 may represent a fuel cell, thermal electric generator, piezo electric charger, solar charger, ultra-capacitor, or other existing or developing power storage technologies. The logic engine 210 preserves the capacity of the battery 208 by reducing unnecessary utilization of the wireless earpieces 202 in a full-power mode when there is little or no benefit to the user (e.g., the wireless earpieces 202 are sitting on a table or temporarily lost). The battery 208 or power of the wireless earpieces are preserved for when being worn or operated by the user. As a result, user satisfaction with the wireless earpieces 202 is improved and the user may be able to set the wireless earpieces 202 aside at any moment knowing battery life is automatically preserved by the logic engine 210 and functionality of the wireless earpieces 202.

In addition, the battery 208 may use just enough power for the transceiver 216 for communicating across a distance separating users of the wireless earpieces 202. Preserving the battery 208 may be as important as tinnitus frequency filtering and treatment may be performed for a user in his/her daily life to alleviate and treat tinnitus.

The logic engine 210 is the logic controlling the operation and functionality of the wireless earpieces 202. The logic engine 210 may include circuitry, chips, and other digital logic. The logic engine 210 may also include programs, scripts, and instructions implemented to operate the logic engine 210. The logic engine 210 may represent hardware, software, firmware, or any combination thereof. In one embodiment, the logic engine 210 may include one or more processors. The logic engine 210 may also represent an application specific integrated circuit (ASIC) or field programmable gate array (FPGA). In one embodiment, the logic engine 210 may execute instructions to manage the wireless earpieces 202 including interactions with the components of the wireless earpieces 202, such as the user interface 214, transceiver 216, and sensors 217.

The logic engine 210 may utilize data and measurements from the transceivers 216 and sensors 217 to determine whether the wireless earpieces 202 are being utilized by different users. For example, distance, biometrics, user input, and other application information, data, and measurements may be utilized to determine whether a tinnitus diagnostic or treatment mode are appropriate or being implemented by the logic engine 210 and other components of the wireless earpieces 202. The logic engine 210 may control actions implemented in response to any number of measurements from the sensors 217, the transceiver 216, the user interface 214, or the physical interface 215 as well as user preferences entered or other default preferences. For example, the logic engine 210 may initialize a tinnitus diagnostic and treatment mode in response to any number of factors, conditions, parameters, measurements, data, values, or other information specified within the user preferences or logic. The logic engine 210 may control the various components of the wireless earpieces 202 to implement the tinnitus diagnostic and treatment modes.

The logic engine 210 may implement any number of processes for the wireless earpieces 202, such as facilitating communications, listening to music, tracking biometrics or so forth. The wireless earpieces 202 may be configured to work together or completely independently based on the needs of the users. For example, the wireless earpieces 202 may be used by two different users at one time.

The logic engine 210 may also process user input to determine commands implemented by the wireless earpieces 202 or sent to the first audio source 204 through the transceiver 216. Specific actions may be associated with user input (e.g., voice, tactile, orientation, motion, gesture, etc.). For example, the logic engine 210 may implement a macro allowing the user to associate frequently performed actions with specific commands/input implemented by the wireless earpieces 202.

In one embodiment, a processor included in the logic engine 210 is circuitry or logic enabled to control execution of a set of instructions. The processor may be one or more microprocessors, digital signal processors, application-specific integrated circuits (ASIC), central processing units, or other devices suitable for controlling an electronic device including one or more hardware and software elements, executing software, instructions, programs, and applications, converting and processing signals and information, and performing other related tasks.

The memory 212 is a hardware element, device, or recording media configured to store data or instructions for subsequent retrieval or later access. The memory 212 may represent static or dynamic memory. The memory 212 may include a hard disk, random access memory, cache, removable media drive, mass storage, or configuration suitable as storage for data, instructions, and information. In one embodiment, the memory 212 and the logic engine 210 may be integrated. The memory 212 may use any type of volatile or non-volatile storage techniques and mediums. The memory 212 may store information related to the status of a user, wireless earpieces 202, first audio source 204, and other peripherals, such as a tablet, smart glasses, a smart watch, a smart case for the wireless earpieces 202, a wearable device, and so forth. In one embodiment, the memory 212 may display instructions, programs, drivers, or an operating system for controlling the user interface 212 including one or more LEDs or other light emitting components, speakers, tactile generators (e.g., vibrator), and so forth. The memory 212 may also store thresholds, conditions, signal or processing activity, proximity data, and so forth.

The transceiver 216 is a component comprising both a transmitter and receiver which may be combined and share common circuitry on a single housing. The transceiver 216 may communicate utilizing Bluetooth, Wi-Fi, ZigBee, Ant+, near field communications, wireless USB, infrared, mobile body area networks, ultra-wideband communications, cellular (e.g., 3G, 4G, 5G, PCS, GSM, etc.), infrared, or other suitable radio frequency standards, networks, protocols, or communications. In one embodiment, the transceiver 216 may be a hybrid or multi-mode transceiver supporting several different communications with distinct devices simultaneously. For example, the transceiver 216 may communicate with the first audio source 204 or other systems utilizing wired interfaces (e.g., wires, traces, etc.), NFC, or Bluetooth communications as well as with the other wireless earpiece utilizing NFMI. The transceiver 216 may also detect amplitudes and signal strength to infer distance between the wireless earpieces 202 as well as the first audio source 204.

The components of the wireless earpieces 202 may be electrically coupled utilizing any number of wires, contact points, leads, busses, wireless interfaces, or so forth. In addition, the wireless earpieces 202 may include any number of computing and communications components, devices, or elements which may include busses, motherboards, printed circuit boards, circuits, chips, sensors, ports, interfaces, cards, converters, adapters, connections, transceivers, displays, antennas, and other similar components. The physical interface 215 is a hardware interface of the wireless earpieces 202 for connecting and communicating with the first audio source 204 or other electrical components, devices, or systems.

The physical interface 215 may include any number of pins, arms, or connectors for electrically interfacing with the contacts or other interface components of external devices or other charging or synchronization devices. For example, the physical interface 215 may be a micro USB port. In one embodiment, the physical interface 215 is a magnetic interface automatically coupling to contacts or an interface of the first audio source 204. In another embodiment, the physical interface 215 may include a wireless inductor for charging the wireless earpieces 202 without a physical connection to a charging device. The physical interface 215 may allow the wireless earpieces 202 to be utilized when not worn as a remote microphone and sensor system (e.g., seismometer, thermometer, light detection unit, motion detector, etc.). For example, measurements, such as noise levels, temperature, movement, and so forth may be detected by the wireless earpieces even when not worn. The wireless earpieces 202 may be utilized as a pair, independently, or when stored in a smart case. Each of the wireless earpieces 202 may provide distinct sensor measurements as needed. In one embodiment, the smart case may include hardware (e.g., logic, battery, transceiver, etc.) to integrate as part of a mesh network. For example, the smart case may be utilized as a node or relay within a mesh network for sending and receiving communications.

The user interface 214 is a hardware interface for receiving commands, instructions, or input through the touch (haptics) of the user, voice commands, or predefined motions. The user interface 214 may further include any number of software and firmware components for interfacing with the user. The user interface 214 may be utilized to manage and otherwise control the other functions of the wireless earpieces 202 including mesh communications. The user interface 214 may include the LED array, one or more touch sensitive buttons or portions, a miniature screen or display, or other input/output components (e.g., the user interface 214 may interact with the sensors 217 extensively). The user interface 214 may be controlled by the user or based on commands received from the first audio source 204 or a linked wireless device. In one embodiment, sharing modes and processes may be controlled by the user interface, such as recording communications, receiving user input for communications, sharing biometrics, queuing communications, sending communications, receiving user preferences for the communications, and so forth. The user interface 214 may also include a virtual assistant for managing the features, functions, and components of the wireless earpieces 202.

The user interface 214 may also include any number of speakers. In one embodiment, the speakers may be utilized to play audio content to the user. Although not shown, the one or more speakers may include several speaker components (e.g., signal generators, amplifiers, filters, drivers, and other circuitry) configured to generate sounds waves at distinct frequency ranges (e.g., bass, woofer, tweeter, midrange, etc.) or to vibrate at specified frequencies to be perceived by the user as sound waves. The speakers of the user interface 214 as well as the logic engine 210 may be specifically tuned to not play the tinnitus frequencies 222 associated with the user. Similarly, the speakers may be utilized to play frequencies, tones, or patterns minimizing the tinnitus suffered by an individual user.

In one embodiment, the user may provide user input for the user interface 214 by tapping a touch screen or capacitive sensor once, twice, three times, or any number of times. Similarly, a swiping motion may be utilized across or in front of the user interface 214 (e.g., the exterior surface of the wireless earpieces 202) to implement a predefined action. Swiping motions in any number of directions or gestures may be associated with specific activities or actions, such as play music, pause, fast forward, rewind, activate a virtual assistant, listen for commands, report biometrics, enabled tinnitus diagnostics and treatment, and so forth.

The swiping motions may also be utilized to control actions and functionality of the first audio source 204 or other external devices (e.g., smart television, camera array, smart watch, etc.). The user may also provide user input by moving his head in a direction or motion or based on the user's position or location. For example, the user may utilize voice commands, head gestures, or touch commands to change the processes implemented by the wireless earpieces 202 as well as the processes executed, or content displayed by the first audio source 204. The user interface 214 may also provide a software interface including any number of icons, soft buttons, windows, links, graphical display elements, and so forth.

In one embodiment, the sensors 217 may be integrated with the user interface 214 to detect or measure the user input. For example, infrared sensors positioned against an outer surface of the wireless earpieces 202 may detect touches, gestures, or other input as part of a touch or gesture sensitive portion of the user interface 214. The outer or exterior surface of the user interface 214 may correspond to a portion of the wireless earpieces 202 accessible to the user when the wireless earpieces are worn within the ears of the user.

In addition, the sensors 217 may include pulse oximeters, accelerometers, thermometers, barometers, radiation detectors, gyroscopes, magnetometers, global positioning systems, beacon detectors, inertial sensors, photo detectors, miniature cameras, and other similar instruments for detecting user biometrics, environmental conditions, location, utilization, orientation, motion, and so forth. The sensors 217 may provide measurements or data may be utilized to select, activate, or otherwise utilize the features and components herein described. Likewise, the sensors 217 may be utilized to awake, activate, initiate, or otherwise implement actions and processes utilizing conditions, parameters, values, or other data within the user preferences. For example, the optical biosensors within the sensors 217 may determine whether the wireless earpieces 202 are being worn and when a selected gesture to activate the virtual assistant 218 is provided by the user.

The first audio source 204 may include components similar in structure and functionality to those shown for the wireless earpieces 202. The computing device may include any number of processors, batteries, memories, busses, motherboards, chips, transceivers, peripherals, sensors, displays, cards, ports, adapters, interconnects, and so forth. In one embodiment, the first audio source 204 may include one or more processors and memories for storing instructions. The instructions may be executed as part of an operating system, application, browser, or so forth to implement the features herein described. For example, tinnitus diagnostic, filtering, and treatment processes may be controlled for the first audio source 204 and the wireless earpieces 202 from an application executed by the first audio source 204.

In one embodiment, the wireless earpieces 202 may be magnetically, wirelessly, or physically coupled to the first audio source 204 to be recharged or synchronized or to be stored. In one embodiment, the first audio source 204 may include applications executed to coordinate the tinnitus features of the wireless earpieces 202 between users. For example, the tinnitus diagnostic and treatment enablement or initiation may be selected from the wireless earpieces 202 themselves for an application utilized by the first audio source 204 to communicate with the wireless earpieces 202. Separate applications executed by the wireless earpieces 202 and the first audio source 204 may function as a single application to enhance functionality, interface and interact, and perform the processes herein described.

The first audio source 204 may be utilized to adjust the user preferences including settings, thresholds, activities, conditions, environmental factors, and so forth utilized by the wireless earpieces 202 and the first audio source 204. For example, the first audio source 204 may utilize a graphical user interface allowing the user to more easily specify any number of conditions, values, measurements, parameters, and factors utilized to perform communications and share content between the wireless earpieces 202.

In another embodiment, the first audio source 204 may also include sensors for detecting the location, orientation, and proximity of the wireless earpieces 202 to the first audio source 204. The wireless earpieces 202 may turn off communications to the first audio source 204 in response to losing a status or heart beat connection to preserve battery life and may only periodically search for a connection, link, or signal to the first audio source 204 or the other wireless earpiece(s). The wireless earpieces 202 may also turn off components, enter a low power or sleep mode, or otherwise preserve battery life in response to no interaction with the user for a period, no detection of the presence of the user (e.g., touch, light, conductivity, motion, etc.), or so forth.

As originally packaged, the wireless earpieces 202 and the first audio source 204 may include peripheral devices such as charging cords, power adapters, inductive charging adapters, solar cells, batteries, lanyards, additional light arrays, speakers, smart case covers, transceivers (e.g., Wi-Fi, cellular, etc.), or so forth. In one embodiment, the wireless earpieces 202 may include a smart case (not shown). The smart case may include an interface for charging the wireless earpieces 202 from an internal battery as well as through a plugged connection. The smart case may also utilize the interface or a wireless transceiver to log utilization, biometric information of the user, and other information and data. The smart case may also be utilized as a repeater, a signal amplifier, relay, or so forth between the wireless earpieces 202 or as part of a mesh network (e.g., a node in the mesh network).

The therapeutic filtering logic 220 may act to eliminate the unwanted noise of the tinnitus frequency 222. In one embodiment, the therapeutic filtering logic may include a high-pass filter modifying an audio signal received externally or internally by the wireless earpieces 202 (e.g., from the memory 212, microphones, etc.) or received from the first audio source 204 to modify the signal by eliminating lower frequencies. The therapeutic filtering logic 220 may also utilize a low-pass filter, removing only the high frequencies from the audio signal. The therapeutic filtering logic 220 may also utilize a band-pass filter.

In another embodiment, the transceiver 216 or sensors 217 including microphones may communicate with the logic engine 210 to filter incoming signals allowing only signals within a select range of frequencies to be heard or decoded. The therapeutic filtering logic 220 may also include a shelving filter which increases or decreases audio signals below a set frequency. In other embodiments, an equalizer may also be used to remove the tinnitus frequency 222 from the user's selected audio stream.

As noted, the layout of the internal components of the wireless earpieces 202 and the limited space available for a product of limited size may affect where the sensors 217 may be positioned. The positions of the sensors 217 within each of the wireless earpieces 202 may vary based on the model, version, and iteration of the wireless earpieces 202 design and manufacturing process. In one embodiment, the wireless earpieces 202 may not include all the sensors 217. In addition, instead of infrared, optical, or capacitive sensors, the wireless earpieces 202 may utilize push buttons for receiving user input. The wireless earpieces 202 may also represent headphones.

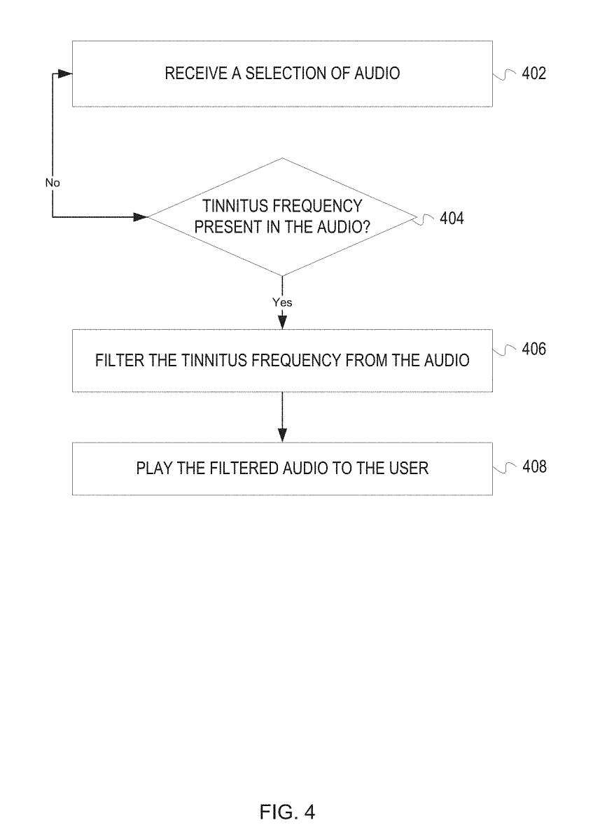

FIG. 3 is a flowchart of a process for associating a tinnitus frequency with a user in accordance with an illustrative embodiment. In one embodiment, the process of FIG. 3 may be implemented by each of the wireless earpieces of a set/pair independently or jointly. In another embodiment, the process of FIG. 4 may be implemented by wireless earpieces in communication with a wireless device or other audio source (jointly the “system”). In another embodiment, the process of FIG. 3 may be implemented by each of the wireless earpieces of a set/pair independently or jointly as part of an audio transparency system able to capture and process ambient sound around the user (discussed in greater detail below). The wireless earpieces and wireless devices described may represent devices, such as those shown in FIGS. 1 & 2.

The process of FIG. 3 may begin by initiating a tinnitus test (step 302). The tinnitus test may be utilized to determine the tinnitus frequencies 222 experienced by a user. The process of FIG. 3 may be performed several times for different users. For example, the wireless earpieces may be utilized by medical professionals for diagnosing and treating several different patients. In one embodiment, the tinnitus test may be initiated automatically in response to placing the wireless earpieces in the ears of the user. In another embodiment, the tinnitus test may be initiated in response to the user opening or selecting a tinnitus testing/diagnostic application executed by the wireless earpieces or an associated wireless device. The tinnitus test may be initiated utilizing any number of automated or manual processes. For example, the tinnitus test may be initiated from an application and corresponding user interface on a smart phone for implementation utilizing the wireless earpieces 202.

Next, the wireless earpieces play tones associated with the tinnitus test to a user (step 304). The tones are played to the user to determine the tinnitus frequency 222 for the user. In one embodiment, the tones may be played in a sequential order. In another embodiment, the tones may be played based on tones historically associated with tinnitus frequencies 222 based on databases, medical observations, user data, and so forth. The tones may be played as part of a program or based on manual user feedback from the user.

Next, the wireless earpieces determine whether a tinnitus frequency 222 is played (step 306). In one embodiment, the tinnitus frequency 222 may be identified by the user. For example, the user may utilize any number of different types of user feedback to identify the tone, such as a verbal affirmation, head nod, tactile input (e.g., tapping or swiping the wireless earpieces), or so forth. The wireless earpieces may also utilize biometric readings to identify the tinnitus frequency 222, such as a reaction (e.g., sweating, head motion, etc.).

If the tinnitus frequency 222 is not played during step 306, the process returns to step 304, and another tone associated with the tinnitus test may be played. If a tinnitus frequency 222 is not found, the process may be manually or automatically terminated.

Next, the wireless earpieces 202 associate the tinnitus frequency 222 with the user (step 308). The tinnitus frequency 222 may be saved in the wireless earpieces 202 or in an associated electronic device 204. For example, a tinnitus testing application executed by one or more of the wireless earpieces 202 and the wireless device 204 may save the tinnitus frequency. The tinnitus frequency 222 may be utilized for subsequent treatments or any number of other processes.

Next, the wireless earpieces 202 distribute the tinnitus frequency 222 (step 308). In one embodiment, the tinnitus frequency 222 may be distributed to one or more devices associated with the user. In another embodiment, the tinnitus frequency 222 may be communicated to medical professionals associated with the user. The tinnitus frequency 222 may also be distributed according to user preferences.

FIG. 4 is a flowchart of a process for treating tinnitus in accordance with an illustrative embodiment. The process of FIG. 4 may be implemented after the process of FIG. 3, simultaneously, concurrently or as an integrated process. The process may begin by receiving a selection of audio (step 402). In one embodiment, the audio may be processed and received internally. For example, the audio may be from an internal source, such as a memory of the wireless earpieces. In another embodiment, the audio may be received from an externally coupled device, such as a smart phone communicating with the wireless earpieces through a Bluetooth connection or the audio could be external ambient sound. The audio may be received as discrete messages, real-time content, or so forth.

Next, the wireless earpieces 202 determine whether the tinnitus frequency 222 is present in the audio (step 404). The wireless earpieces 202 may perform frequency or spectrum analysis for the audio to determine if and when the audio includes the tinnitus frequency 222. In one embodiment, the wireless earpieces 202 may analyze and process the audio creating an audio map of the associated audio. For real-time audio, the audio may be analyzed in real-time before being played to the user. In some embodiments, the wireless earpieces 202 may perform analysis on all available audio content to reduce the processing or analysis required in the future.

Next, the wireless earpieces filter the tinnitus frequency from the audio (step 406). In one embodiment, the audio may be filtered and then saved as filtered audio. In another embodiment, the audio may be filtered in real-time. Analysis including an audio map, markers, flags, metadata, or so forth may be utilized by the wireless earpieces 202 to perform the filtering or generate the filtered audio during step 406.

Next, the wireless earpieces 202 play the filtered audio to the user (step 408). The audio is played without the tinnitus frequency 222. In one embodiment, the filtered audio may be referred to as a therapeutic audio stream. The audio is filtered during the process of FIG. 4 to aid in the user's tinnitus therapy process. As the tinnitus frequency is removed from the audio, the other frequencies in the filtered audio stimulate the user's neurons which neighbor the tinnitus neuron. The stimulation of other neurons reduces the activity in the neurons response for the tinnitus experienced by the user. The filtered audio reduces the symptoms of tinnitus by not activating the neurons associated with tinnitus. As a result, the user may have alleviated symptoms or symptoms eventually go away altogether.

In one embodiment, the user may track the utilization of the tinnitus treatments performed during FIG. 4 to provide analysis and details to the user. For example, the wireless earpieces may show reports indicating how often tinnitus frequencies are filtered. The blocking of the tinnitus frequency as described in FIG. 4 may also be performed for everyday sounds the user experiences. For example, the wireless earpieces may have an audio processing or audio transparency feature filtering all audio typically heard by the user. The wireless earpieces may receive all audio from an environment through one or more microphones. The audio may then be processed to perform noise reduction, hearing protection, and tinnitus frequency treatments as described in FIG. 4.

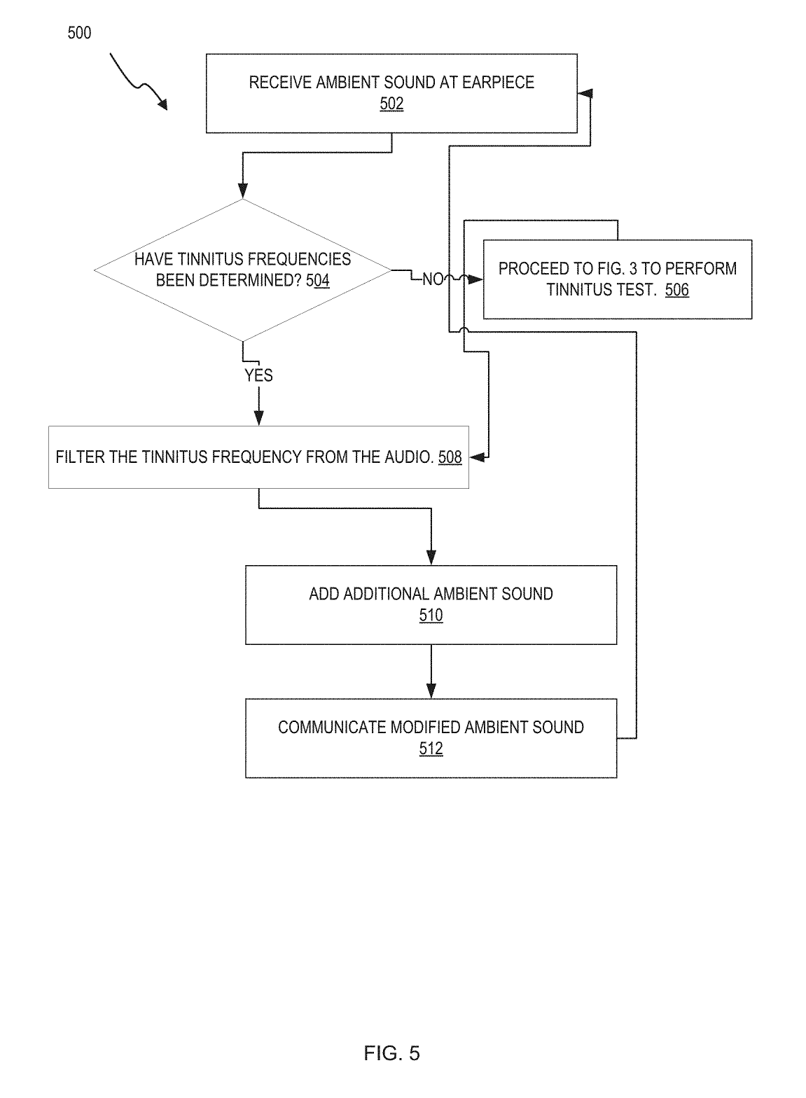

FIG. 5 illustrates a flowchart of one embodiment of a method 500 of adding an ambient environment sound and treating tinnitus in accordance with an embodiment of the present invention. In step 102, sound is received at the earpiece. The sound in step 502 could be ambient sound and is being processed as part of an audio transparency system allowing the user to capture the sounds around him(er) or the sound could be any audio received by wireless earpieces 202. However, for purposes of FIG. 5, the sound will be discussed as ambient sound.

One or more microphones of the earpiece 202 may be used to detect the ambient sound. It is also to be understood there may be a set of wireless earpieces present including a left earpiece and a right earpiece and each earpiece may have one or more microphones. At step 504 it is determined if the tinnitus frequencies 222 have been determined and set. If the tinnitus frequencies are still unknown and/or it has been greater than six months, for example, since the user was tested, method 500 proceeds to step 302 of FIG. 3 to initiate a tinnitus test at step 506. Once the tinnitus frequencies 222 are determined, method 500 proceeds to step 508 to filter the tinnitus frequencies 222 from the ambient sound. This filtering can be performed with most any analog and/or digital sound filtering such as the filtering discussed in detail above.

In step 510, the ambient sound is processed to mix in additional sound selected by the user. The additional sound selected by the user may be of any number of types. It may be ambient sound of a type which the user may finding soothing or relaxing such as waves at a beach, waterfalls, or similar natural sounds. It may be white noise if the user finds it helpful to drown out the other ambient noise. It may be the sound of a busy street, or any other type of ambient environment the user wishes to add. It should be understood, although sound is added, the original ambient sound is still present although it may be processed to a modified form. Thus, the user may still hear various sound sources within the user's environment.

In one embodiment, the additional ambient sounds may be stored as one or more sound files on the wireless earpiece and the processor may use a mixing function to mix the additional ambient sounds with the detected ambient sounds. In some instances, the additional ambient sounds may be generated by the processor instead of being stored. The user may select the additional ambient sounds in any number of ways. For example, voice commands such as may be detected using one or more bone conduction microphones may instruct the earpiece as to which ambient sound(s) to add. The gestural control interface may be used to instruct the earpiece as to which ambient sound(s) to add. Where the earpiece includes a wireless transceiver, the earpiece may be in operative communication with other devices such as with a mobile device and the user may use the mobile device to instruct the earpiece which ambient sounds to add.

In step 512, the modified ambient sound which includes the ambient sound detected as well as the additional ambient sound communicated to one or more speakers to transduce or produce the modified ambient sound. Thus, the user hears the modified ambient sound. It is to be understood the processor may provide for generating 3D sound, so a user perceives sounds as being from a 3D environment. The added ambient sound may be reproduced as 3D sound. Method 500 can be iterative and step 502 would be run again quickly after step 512.

Wireless earpieces configured to diagnose, administer, and track a tinnitus therapy have been shown and described herein. The sound and other audio content may be filtered in real-time or filtered and saved. In one embodiment, determining a tinnitus frequency associated with a user may be saved, logged, or distributed. A tinnitus application executed by the wireless earpieces, an associated electronic device, or both may track the progress of the user. The tinnitus application may receive user input to determine the tinnitus frequency, symptom severity, progress towards symptom alleviation through filtering, and so forth. In addition, utilization of audio filtering to reduce the user's tinnitus symptoms may be tracked. For example, the amount and times during which the tinnitus frequency is filtered may be saved. Although the methods and systems displayed in this application are contained within the wireless earpieces, other embodiments have also been considered. Wireless earpieces may connect to an outside source such an audiogram or other tonal test to diagnose a user's specific tinnitus frequency. The wireless earpieces may also connect to another electronic device for filtering out the tinnitus frequency from an audio stream. Other devices providing libraries of music and filtered audio streams may connect to the user's wireless earpieces, allowing the user to store a larger portion of filtered audio elsewhere while storing select audio streams on the earpieces. Although specific embodiments shown and described herein, the illustrative embodiments contemplate numerous variations and additions.

The features, steps, and components of the illustrative embodiments may be combined in any number of ways and are not limited specifically to those described. The illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated other alternatives or exemplary aspects are considered included in the disclosure. The description is merely examples of embodiments, processes or methods of the invention. It is understood any other modifications, substitutions, and/or additions may be made, which are within the intended spirit and scope of the disclosure. For the foregoing, it can be seen the disclosure accomplishes at least all the intended objectives.

The previous detailed description is of a small number of embodiments for implementing the invention and is not intended to be limiting in scope. The following claims set forth several the embodiments of the invention disclosed with greater particularity.

What is claimed is:

1. A method for treating tinnitus utilizing wireless earpieces, comprising:
receiving a selection of audio through the wireless earpieces;
determining whether a tinnitus frequency associated with a user wearing the wireless earpieces is present in the audio;
filtering the tinnitus frequency from the audio to generate filtered audio; and
playing the filtered audio to the user utilizing the wireless earpieces.

2. The method of claim 1, further comprising:
initiating a tinnitus test for the user;
playing tones associated with the tinnitus test to the user;
determining whether the tinnitus frequency is played to the user;
associating the tinnitus frequency with the user in response to determining the tinnitus frequency was played to the user.

3. The method of claim 2, wherein the tinnitus test is pre-saved in the wireless earpieces.

4. The method of claim 2, wherein the tinnitus test is performed a plurality of times for a plurality of users utilizing the wireless earpieces.

5. The method of claim 2, wherein the tinnitus test is received from a wireless device in communication with the wireless earpieces.

6. The method of claim 1, wherein the selection is one or more of communications, music, entertainment, or audio recorded by microphones of the wireless earpieces.

7. The method of claim 1, wherein the audio is processed in real-time.

8. The method of claim 1, wherein the tinnitus frequency is attenuated.

9. The method of claim 1, further comprising:
distributing the tinnitus frequency to one or more devices in communication with the wireless earpieces.

10. The method of claim 1, further comprising:
tracking filtering of the tinnitus frequency performed by the wireless earpieces.

11. The method of claim 1, wherein the wireless earpieces include one or more biometric sensors and a transceiver for communicating with one or more wireless devices.

12. The method of claim 1, further comprising:
receiving user input confirming the tinnitus frequency.

13. The method of claim 1, further comprising:
identifying the user utilizing the wireless earpieces.

14. A wireless earpiece, comprising:
a housing for fitting in an ear of a user;
a logic engine controlling functionality of the wireless earpiece;
a plurality of sensors reading user input from the user;
a transceiver communicating with at least a wireless device;
wherein the logic engine receives a selection of audio through the plurality of sensors, determines whether a tinnitus frequency associated with a user wearing the wireless earpiece is present in the audio, filters the tinnitus frequency from the audio to generate filtered audio, and plays the filtered audio to the user utilizing one or more speakers.

15. The wireless earpiece of 14, wherein the logic engine initiates a tinnitus test for the user, plays tones associated with the tinnitus test to the user, determines whether the tinnitus frequency is played to the user, and associates the tinnitus frequency with the user in response to determining the tinnitus frequency was played to the user.

16. The wireless earpiece of claim 14, wherein the plurality of sensors receive user input confirming the tinnitus frequency, and wherein the transceiver distributes the tinnitus frequency to at least the wireless device.

17. The wireless earpiece of claim 14, wherein the audio is processed in real-time to perform the filtering.

18. A method for treating tinnitus utilizing wireless earpieces, comprising:
receiving ambient sound through the wireless earpieces;
determining whether a tinnitus frequency has been associated with a user;
initiating a tinnitus test for the user if no tinnitus frequency has been associated with the user;
determining whether the tinnitus frequency associated with a user wearing the wireless earpieces is present in the ambient sound if the tinnitus frequency has been associated with the user; and
filtering the tinnitus frequency from the ambient sound to generate filtered ambient sound.

19. The method of claim 18, further comprising playing the filtered ambient sound to the user utilizing the wireless earpieces.

20. The method of claim 19, further comprising:
playing tones associated with the tinnitus test to the user;
determining whether the tinnitus frequency is played to the user; and
associating the tinnitus frequency with the user in response to determining the tinnitus frequency was played to the user.
